SEQUENCE LISTING (1 sequence)

SEQ ID NO: 1 - DNA, 17 bp, Saccharomyces cerevisiae
  misc_feature (4)..(14): n is a, c, g, or t
cggnnnnnnnnnnnccg

(19) United States
(12) Patent Application Publication
(10) Pub. No.: US 2020/0208165 A1
(43) Pub. Date: Jul. 2, 2020

(54) Synthetic promoters useful for expression in plant cells

(71) Applicant: The Regents of the University of California, Oakland, CA (US)

(72) Inventors: Dominique Loque, Vernier (CH); Patrick M. Shih, Berkeley, CA (US)

(73) Assignee: The Regents of the University of California, Oakland, CA (US)

(21) Appl. No.: 16/816,100

(22) Filed: Mar. 11, 2020

Related U.S. Application Data
(63) Continuation of application No. PCT/US2018/050514, filed on Sep. 11, 2018.
(60) Provisional application No. 62/557,107, filed on Sep. 11, 2017.

(51) Int. Cl. C12N 15/82

(52) U.S. Cl. CPC C12N 15/8223

(57) ABSTRACT

The present invention provides for a genetically modified plant cell or plant, comprising: (a) (i) one or more nucleic acids each encoding one or more transcription factors (or transcription activators) operatively linked to a first tissue-specific or inducible promoter, (ii) one or more nucleic acids each encoding one or more transcription repressors each operatively linked to a second tissue-specific or inducible promoter, or (iii) combinations thereof; and (b) one or more nucleic acids each encoding one or more independent genes of interest (GOI) each operatively linked to a promoter that is activated by the one or more transcription factors (or transcription activators), repressed by the one or more transcription repressors, or a combination of both.

### CROSS REFERENCE TO RELATED APPLICATIONS

This application claims priority as a continuation application of International PCT Patent Application No. PCT/US2018/050514, filed Sep. 11, 2018, which claims priority to U.S. Provisional Patent Application Ser. No. 62/557,107, filed Sep. 11, 2017; both of which are incorporated by reference in their entireties.

### STATEMENT OF GOVERNMENTAL SUPPORT

The invention was made with government support under Contract Nos. DE-AC02-05CH11231 awarded by the U.S. Department of Energy. The government has certain rights in the invention.

### REFERENCE TO SUBMISSION OF A SEQUENCE LISTING

This application includes a Sequence Listing as a text file named “2015_049_03_Sequence_Listing_ST25” created Mar. 11, 2020 and containing 652 bytes. The material contained in this text file is incorporated by reference in its entirety for all purposes.

### FIELD OF THE INVENTION

The present invention is in the field of regulating gene expression in plants.

### BACKGROUND OF THE INVENTION

Plants offer a unique platform to address many imminent challenges that face society, as future engineering efforts hold promise in promoting sustainable agriculture, renewable energy, and green technologies¹. However, the tools to effectively modify and engineer plants are still in their infancy. One major hurdle has been the development of tools and genetic parts that enable precise control of transgene expression in plants. Just as significant progress has been achieved in the field of microbial metabolic engineering through the characterization of reliable genetic parts to regulate gene expression²³, the plant synthetic biology field will require similar advances in defining gene regulatory principles that will ultimately facilitate more complex engineering designs.

Many genetic and metabolic engineering efforts require the robust and accurate control of genes^{4, 5}. Just as a metabolic pathway may need optimization to increase flux or avoid bottlenecks, the ability to modulate gene expression provides an efficient and simple means to address many of these issues. Nonetheless, the vast majority of plant engineering efforts have traditionally been limited to a small number of characterized promoters that have largely relied on a few constitutive promoters, which may result in unintended pleiotropic effects or toxicity issues. Moreover, many classic promoters have been incorrectly labeled as ‘constitutive,’ as many are not expressed in all tissue⁶, nor do they express at similar expression levels across various tissue types. Thus, this broad stroked approach and traditional reliance on ‘constitutive’ promoters represents a major barrier that separates the level of engineering complexity that can be deployed in model microbial versus plant systems.

Another key distinction between plants and unicellular microbes is the drastically different levels of organismal and developmental complexity, best demonstrated by the multitude of tissue types that make up plants. Additionally, many of these different cell types are highly dynamic and respond to their external environment. Thus, a key hurdle has been how to artificially design promoter elements that replicate elements of spatial and temporal control over gene expression. In comparison to microbial efforts, these challenges are unique and specific to plants and have not yet been thoroughly addressed. The majority of previous studies that have required tissue-specific expression of transgenes in plants have utilized endogenous promoters; however, these promoters cannot modulate expression strength, thus demonstrating the one-dimensional limitation of being restricted to these endogenous sequences.

One obstacle that has thwarted plant engineering efforts is the natural phenomenon of epigenetic silencing. Although poorly understood, plants have evolved robust defense mechanisms that may perceive multiple transgenes driven by the same promoter as a threat, resulting in gene silencing at the transcriptional (gene inactivation via DNA methylation) and post-transcriptional (RNA degradation) level⁸. Thus, although many engineering efforts require the coordinated expression of multiple genes, it has long been observed that stacking the same promoter multiple times may also dramatically increase the chance of gene silencing⁹. Hence, plant synthetic biology efforts will need to design promoters that circumvent silencing issues and facilitate the coordinated expression of multiple genes simultaneously in a tissue-specific manner. Taken all together, an ideal system would incorporate a library of synthetic promoters that would 1) control gene expression strength, 2) facilitate tissue-specific expression, and 3) decrease the likelihood of gene silencing mechanisms by avoiding the use of homologous promoter sequences.

### SUMMARY OF THE INVENTION

The present invention provides for a genetically modified plant cell or plant, comprising: (a) (i) one or more nucleic acids each encoding one or more transcription factors (or transcription activators) operatively linked to a first tissue-specific or inducible promoter, (ii) one or more nucleic acids each encoding one or more transcription repressors each operatively linked to a second tissue-specific or inducible promoter, or (iii) combinations thereof; and (b) one or more nucleic acids each encoding one or more independent genes of interest (GOI) each operatively linked to a promoter that is activated by the one or more transcription factors (or transcription activators), repressed by the one or more transcription repressors, or a combination of both.

In some embodiments, the transcription factor (or transcription activator) is eukaryotic or prokaryotic (or bacterial). In some embodiments, the transcription factor (or transcription activator) is synthetic. In some embodiments, the transcription repressor is synthetic. In some embodiments, the transcription factor (or transcription activator) and the transcription repressor are synthetic.

In some embodiments, any one of the transcription factor (or transcription activator), plant cell or plant, one or more of the GOI, any other transcription factor (or transcription activator), transcription repressor, and/or any of the promoters is heterologous to any other member of the list herein. In some embodiments, the transcription factor (or transcription activator) is heterologous to the plant cell or plant, one or more of the GOI, any other transcription factor (or transcription activator), transcription repressor, and/or any of the promoters. In some embodiments, the transcription repressor is heterologous to the plant cell or plant, one or more of the GOI, any other transcription factor (or transcription activator), and/or any of the promoters.

In some embodiments, the genetically modified plant cell or plant comprises: (a) a first nucleic acid encoding a transcription factor (or transcription activator) operatively linked to a first tissue-specific or inducible promoter, (b) optionally a second nucleic acid encoding a transcription repressor operatively linked to a second tissue-specific or inducible promoter; and (c) one or more nucleic acids each encoding one or more independent genes of interest (GOI) each operatively linked to a promoter that is activated by the transcription factor (or transcription activators), repressed by the transcription repressors, or a combination of both.

In some embodiments, the genetically modified plant cell or plant comprises: (a) optionally a first nucleic acid encoding a transcription factor (or transcription activator) operatively linked to a first tissue-specific or inducible promoter, (b) a second nucleic acid encoding a transcription repressor operatively linked to a second tissue-specific or inducible promoter; and (c) one or more nucleic acids each encoding one or more independent genes of interest (GOI) each operatively linked to a promoter that is activated by the transcription factor (or transcription activators), repressed by the transcription repressors, or a combination of both.

Each GOI is operatively linked to a promoter that is activated by the transcription factor (or transcription activator), repressed by the transcription repressors, or a combination of both. In some embodiments, the promoter comprises one or more DNA-binding sites specific for the transcription factor (or transcription activator), one or more DNA-binding sites specific for the transcription repressor, or a combination of both. In some embodiments, the promoter comprises 1, 2, 3, 4, 5, 6, 7, 8, 9, or 10 DNA-binding sites specific for the transcription factor (or transcription activator), 1, 2, 3, 4, 5, 6, 7, 8, 9, or 10 DNA-binding sites specific for the transcription repressor, or a combination of both.

The present invention provides for a library of unique promoters, wherein the promoter strengths of every unique promoter is identified relative to every other unique promoter.

In some embodiments, the library comprises at least 8 unique promoters. In some embodiments, the library comprises at least 10 unique promoters. In some embodiments, the library comprises at least 20 unique promoters. In some embodiments, the library comprises at least 50 unique promoters. In some embodiments, the library comprises at least 100 unique promoters. Each library of unique promoters has had the promoter strength of each promoter tested and compared to every other unique promoter, such that the promoter strengths of every unique promoter is identified relative to every other unique promoter, and the unique promoters can be ordered according to descending or ascending promoter strength.

The present invention provides for a method of constructing the library of unique promoters of the present invention comprising: constructing a series of promoters wherein the first promoter comprises one DNA-binding site specific for the transcription factor (or transcription activator), the second promoter comprises two DNA-binding sites specific for the transcription factor (or transcription activator), the third promoter comprises three DNA-binding sites specific for the transcription factor (or transcription activator), and so on and so forth; optionally a corresponding number of unique promoters with 1, 2, 3, and so on DNA-binding sites specific for the transcription repressor.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. A strategy for repurposing DNA-binding transcriptional regulators for generating synthetic promoters that are functional in any host.

FIG. 2. The Gal4 DNA binding protein is utilized in yeast transcriptional regulation of galactose transcriptional network.

FIG. 3. An embodiment of the invention as applied to plants. A plant is engineered with tissue-specific gene stacking using synthetic gal promoters.

FIG. 4. A strategy for controlling repression of synthetic promoters.

FIG. 5A. Design and characterization of a library of synthetic promoters. Brute force strategy to design and generate a library of promoters with varying expression strengths. Synthetic activators are generated by fusing a Gal4 DNA-binding domain to a nuclear localization sequence and VP16 activator domain. A library of cis-elements that bind Gal4 and vary in sequence were gathered from endogenous yeast promoters that fall within the Gal regulon. Various plant minimal promoters were also gathered. Five random cis-elements were concatenated in front of a minimal promoter in order to design and generate synthetic promoters.

FIG. 5B. Design and characterization of a library of synthetic promoters. Synthetic promoters were characterized by fusing them in front of a GFP. A constitutive MAS promoter was used to drive a DsRed in order to normalize between samples. Synthetic activators were driven by the constitutive Actin promoter, enabling the expression of the GFP. A control construct was generated, lacking the synthetic activator, allowing the measurement of basal expression of the synthetic promoters.

FIG. 5C. Design and characterization of a library of synthetic promoters. A range of expression strengths can be observed with the designed synthetic promoters. Constructs including the synthetic activator enable GFP expression (blue), while controls lacking synthetic activators provide basal expression levels of synthetic promoters (red). Constructs were transiently expressed in N. benthamiana leaves, and GFP fluorescence was normalized to constitutive expression of DsRed and reported in arbitrary units.

FIG. 6A. Utilizing synthetic promoters for the coordinated expression of multiple stacked transgenes. Schematic of how synthetic activator can be utilized to drive the concerted expression of multiple downstream genes of interest in a spatial or temporal specific manner. Cartoon demonstrates the basic design of constructs used to demonstrate how the expression of multiple transgenes (GFP, DsRed, and GUS) can be controlled by regulation of the synthetic activator.

FIG. 6B. Utilizing synthetic promoters for the coordinated expression of multiple stacked transgenes. Spatial regulation of multiple reporter genes under the control of the seed-specific expression of the synthetic activator driven by the At2S3 promoter. Seeds from transgenic plants show expression of all three reporter genes, whereas vegetative tissue taken from roots or whole seedling showed indication of reporter gene expression.

FIG. 6C. Utilizing synthetic promoters for the coordinated expression of multiple stacked transgenes. Temporal regulation of multiple reporter genes under the control of the synthetic activator which responds to environmental stimuli. The AtPht1.1 promoter is driving the synthetic activator, enabling the inducible expression of all three downstream reporter transgenes to be turned on in response to phosphate deprivation.

FIG. 7A. Utilizing synthetic repressors enable synthetic promoter compatibility with repressor logic. Schematic of constructs used to demonstrate how the synthetic repressor inhibits and competes with the synthetic activator to repress expression of a given transgene. Samples above the grey dashed line are only expressing the synthetic activator, whereas samples below the dashed line have included expression of the synthetic repressor.

FIG. 7B. Utilizing synthetic repressors enable synthetic promoter compatibility with repressor logic. Labeling the various constructs infiltrated into a leaf with and without the synthetic repressor. Two different synthetic promoters (one high and one medium expression strength) were used to demonstrate the effects of the synthetic repressor. DsRed expression is constitutively driven by the nos promoter to enable normalization. Infiltration of a construct only expressing the synthetic repressor was used as a negative control.

FIG. 7C. Utilizing synthetic repressors enable synthetic promoter compatibility with repressor logic. GFP expression controlled by two different synthetic promoters. Spots infiltrated without the repressor are above the grey dashed line, whereas the synthetic repressor was also co-infiltrated in samples below the dashed line. Samples within dashed yellow lines correspond to each other as with and without the synthetic repressor.

FIG. 7D. Utilizing synthetic repressors enable synthetic promoter compatibility with repressor logic. Constitutive expression of DsRed allows for an internal control and normalization of GFP expression, as it is generally expressed at the same level in all samples.

FIG. 7E. Utilizing synthetic repressors enable synthetic promoter compatibility with repressor logic. Quantification of the amount of repression observed with the introduction of the synthetic repressor in conjunction with constructs already expressing a synthetic activator driving GFP expression. Samples are normalized to DsRed expression.

FIG. 8A. Teasing apart the additive effect of DNA elements reveals generalizable trends in promoter expression strength. Overall design of every combination of concatenated cis-elements and minimal promoters in order to test if individual parts additively contribute to expression strength in a predictable manner. Therefore, expected promoter strengths will increase with the addition of strong cis-elements and minimal promoters, whereas weak expression would be expected when using weak cis-elements and minimal promoters.

FIG. 8B. Teasing apart the additive effect of DNA elements reveals generalizable trends in promoter expression strength. Characterization of every combination of synthetic promoters generated from five cis-elements and five minimal promoters spanning expression strengths. Promoter strength is measured based on GFP fluorescence and normalized to constitutively expressed DsRed, in the same construct design described in FIG. 4. The general tendency of strong expression being correlated with the usage of DNA elements that promote expression is observed; however, some noise is still observed, which can be due to the context dependence of promoter elements.

FIG. 8C. Teasing apart the additive effect of DNA elements reveals generalizable trends in promoter expression strength. Qualitative characterization of five synthetic promoters demonstrating the trend of increasing promoter strength with usage of DNA elements with cis-elements and minimal promoters that promote higher expression. Five constructs (labeled and outlined in yellow) which span expression strengths varying expression strengths were infiltrated into a N. benthamiana leaf and imaged.

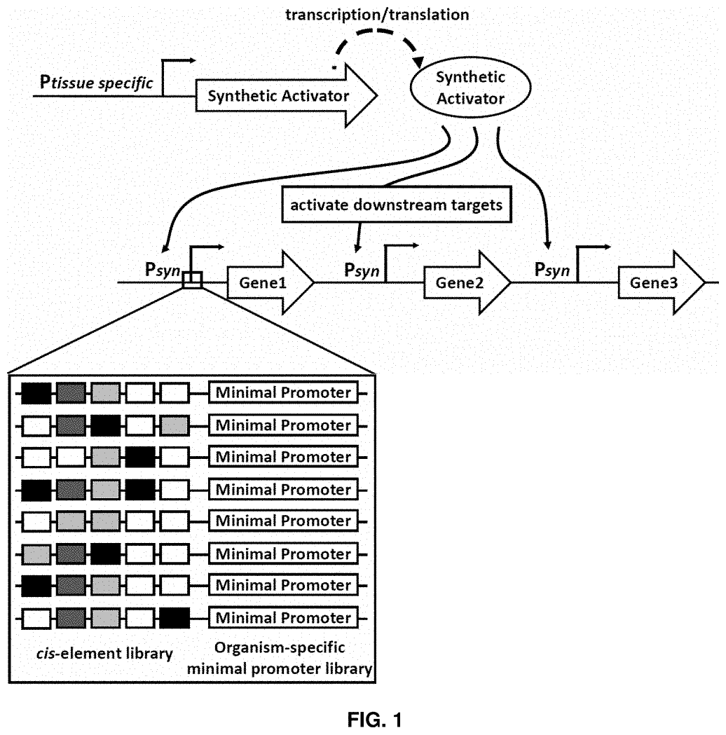

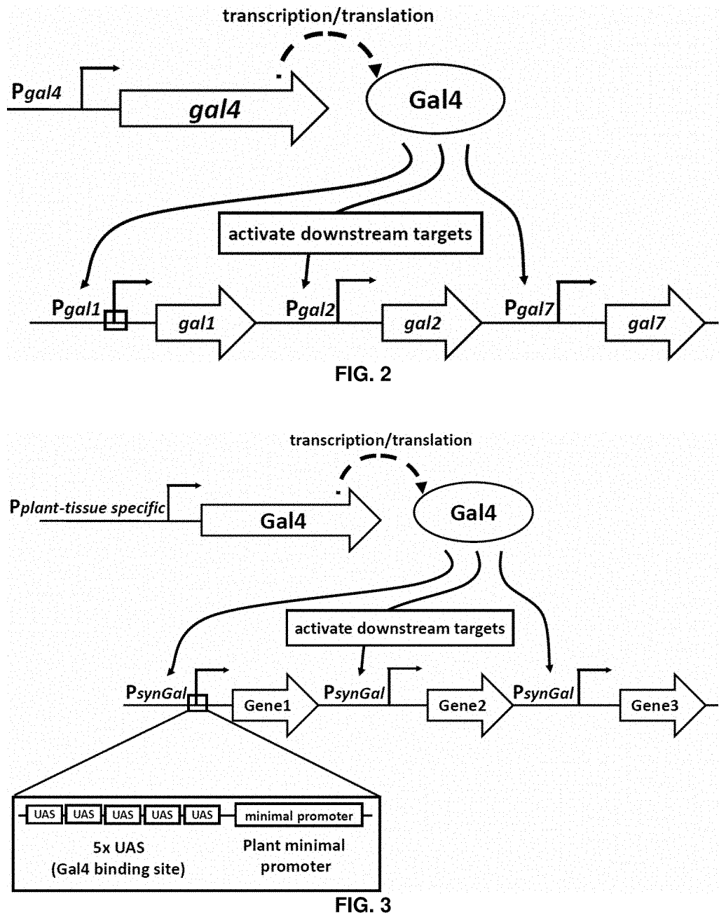

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

A major problem in new or non-model organisms is the controlled expression of multiple genes in a certain manner. This problem is compounded if one is trying to express multiple genes simultaneously. Moreover, expression of all these genes in some temporal or spatial manner but not in other conditions is essentially impossible without adding an additional level of complexity. This is a limiting factor in many biotechnologically relevant organisms, such as plants. Technical challenges that were overcome to make this invention include generation of synthetic promoter sequences, generation of libraries of varying expression strength, validating the varying expression strengths, and the design, creation, and characterization of synthetic Transcription Factors and synthetic Transcriptional Repressors. Currently, efforts to control expression strength in plants are limited to merely using strong constitutive promoters or using multiple copies of tissue-specific promoters. The ladder approach is not adequate as runs the risk of problems of gene silencing or gene recombination.

The present invention provides for a strategy to design system utilizing synthetic promoters for the ultimate purpose of controlling expression strength, tissue-specificity, and environmentally-responsive promoters and associated downstream products (e.g. RNA, protein). This method utilizes any DNA-binding protein (synthetic Transcriptional Activator; sTA) with its corresponding DNA binding sequence (cis-element), where multiple slightly varying nucleotide sequences of cis-elements are concatenated to provide variability in the binding strength of the transcriptional regulator. The cis-elements are fused to varying minimal promoter sequences (minimal promoter or minimal promoter+UTR upstream sequence of ATG) of the eukaryote host organism of interest to enable the synthetic Transcriptional Activator the ability to control expression of the target downstream gene. This invention is novel in that it provides a strategy for engineering an entirely orthogonal transcriptional network into any eukaryotic host for controlling expression strengths of multiple genes through the heterologous expression of one transcriptional regulator. The strategy and method of designing a library of synthetic promoters is also novel.

This invention enables one skilled in the art to control the expression of a single or multiple genes simultaneously in any eukaryote organism with only one endogenous promoter. Many times, such as in plants, reuse of the same promoter to drive heterologous expression of multiple genes may increase the likelihood of gene silencing and even creates genome instability. Moreover, use of one endogenous promoter may offer the desired expression level required to express a gene of interest. This invention offers the capacity of retaining expression specificity while offering a dynamic range of expression of the transgene. For example, there are many promoters that display tissue-specific expression in one specific tissue (e.g., plant roots, seeds, leaves, or the like); however, there is no rational way of amplifying or decreasing the expression strength of any given promoter. By utilizing the promoter of interest to drive a sTA, one can generate a library of synthetic promoters that are turned on by the sTA at varying expression strengths. This is a much more efficient and productive way in controlling the exact expression strength of a single or multiple genes in a tissue-specific or environmentally-responsive manner.

Current systems that are not as developed as microbes, such as E. coli and yeast, lack the characterized promoters necessary to adequately manipulate genes for metabolic engineering efforts. Using current methods, one either reuses the same promoter or run the risk of gene silencing or gene recombination problems. Current methods are laborious and tedious as one must first characterize new sets of promoters in a new host organisms. The present invention addresses all these issues simultaneously by utilizing synthetic promoters.

The present invention can be applied to any host eukaryotic organism of interest. For example, if five enzymes are necessary to reconstitute a heterologous metabolic pathway in the roots of ‘Plant A’, using the conventional methods, five promoters would need to be characterized in to express genes in roots of ‘Plant A.’ These promoters may not work for expression in ‘Plant B’ (e.g. they may not retain same expression profile or strength, or even be functional). The synthetic promoters of the present invention would remain the same between both Plant A and B, as only one promoter would be needed to drive the Synthetic Transcriptional Regulator in either plant to ultimately control expression of all five downstream enzymes. The synthetic promoters of the present invention can be used directly, or with minimal routine modification, in other eukaryotic cells, such as fungi and animal cells. The present invention is a significant advancement in controlling expression of heterologous genes and metabolic pathways in less characterized biological organisms.

This invention offers the ability to perform various permutations and test multiple expression profiles. For example, one set of plants could be generated with different promoters driving the sTA (set A) and another set of plants would be transformed with different combination of synthetic promoters driving one or a multiple transgene of interests (set B. Plants from set A could be crossed with those of set B, this would great a 2D matrix of new plants expressing transgene of interests in different tissues and at different strength. This approach has the capacity to reduce number of transformations. For example, generation of 50 plants for each set (A and B) will require 100 transformations and will be used to generate 2500 combinations that would normally require 2500 independent transformations without the use of matrix as presented above. Such matrix approach is applicable to any eukaryotic host that can be crossed such as crops and yeast.

The present invention provides for a strategy to repress genes of interest expressed by synthetic promoters. This invention works in conjunction with our previous ROI which describes the development of synthetic promoters to control expression strength, tissue-specificity, and environmental responsiveness in multiple genes in tandem. The invention described here provides an additional layer of control and regulation by utilizing a synthetic Transcriptional Repressor to repress expression of genes. A DNA-binding domain which binds the synthetic promoter cis elements have a fused repressor domain attached. There are varying strategies to control the level of repression. For example, we have shown that various derivatives of fusion proteins (N- or C-terminus) can result in varying levels of repression. Furthermore, repressors could also either be degrade, sequestered, or change in protein conformation to control spatial and temporal changes in repression of genes of interest. Our previous ROI allows for the control of expression strength using synthetic promoters. Here, this current ROI permits control of expression strength in some tissues, but then the repression of those genes in others. This is incredibly important for more sophisticated engineering strategies for multicellular organisms, especially crop plants. This invention is novel by further elaborating on an already novel approach to developing synthetic promoters. Most efforts in the field have focused on controlling the activation and expression of genes. Conversely, we have developed a method to use synthetic Transcriptional Repressors on synthetic promoters to control repression of genes.

With the synthetic Transcriptional Repressors of this present invention, one skilled in the art is able to subtract out certain tissues for where one or more genes of interest (GOI) are expressed. For example, one can use a constitutive promoter to activate expression of GOIs in all tissue and express a repressor specifically in the roots; thus, only expression will be found in the shoots. This is useful for those who may want to avoid the length and laborious process of discovering, characterizing, and validating promoters that have properties they want. Furthermore, within the context of the synthetic promoters system, this provides an additional level of regulation which other strategies and technologies do not have. A further application of this invention is in the context of an environmental response. For example, if one desires a GOI to be repressed in response to an abiotic or biotic stress for optimal growth, the present invention can provide for a repression system to effect a gradual decrease in expression of the GOIs.

The present invention provides for any eukaryotic host controlling multiple genes through repression by use of synthetic Transcriptional Activators and/or synthetic Transcriptional Repressors. The present invention provides for a further layer of regulation by the synthetic promoters of this invention. This platform can be used to control expression of GOIs in less characterized organisms used in biotechnology.

The present invention provides for the engineering of any DNA binding protein to design synthetic promoters in a heterologous organism. In some embodiments, the DNA binding protein is any repressor polypeptide having at least 70% amino acid identity with EilR, SmvR, KmrR, RcdA, or QacR as disclosed in Ruegg et al. 2014. (Ruegg et al. 2014, Nature Communications, 5, 3490) and Ruegg (U.S. Patent Application Publication No. 2017/0002363 A1, which is hereby incorporated by reference), which is incorporated by reference. This protein was selected for it flexible binding site composed of 20-24 nucleotides (inverted repeat sequence) that is similar flexibility to that of Gal4 system.

U.S. Patent Application Publication No. 2017/0002363 A1 teaches the use of synthetic transcription activators that can be regulated by a specific chemical, such as EilR DNA binding activity, that can be chemically blocked by a hydrophobic inducer, such as a hydrophobic cation inducer, such as a triarylmethane, acridine, phenazine, phenothiazine, or xanthene, or a hydrophobic anion inducer.

This invention allows one skilled in the art to make a synthetic activator and set of promoters out of any DNA transcription factor, such as a eukaryotic transcription factor, such as Gal4, or a bacterial DNA binding protein, such as EilR DNA binding protein, and other related DNA binding proteins such as SmvR, KmrR, RcdA, or QacR. This invention allows one skilled in the art to design any synthetic promoter from any DNA binding protein that can also be chemically regulated, such as EilR, which has not been previously described.

The present invention can be extended to any eukaryotic system. In some embodiments, the synthetic transcription factor is a eukaryotic transcription factor, or an engineered form thereof. In some embodiments, the synthetic transcription factor is a bacterial DNA binding repressor protein modified into a synthetic transcriptional activator in a eukaryotic system within the present invention. In some embodiments, the bacterial protein can be allosterically regulated by a novel compound, which can add additional layers of regulation.

This invention can be used by nearly any biotechnology industry. This invention can easily be utilized for any eukaryotic host, such as plant, yeast or animal hosts.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

Many strategies in agricultural biotechnology require the precise and coordinated control of multiple genes in order to effectively modify complex plant traits. Thus, engineering efforts have been hindered by the lack of characterized promoters that allow for reliable and targeted expression of transgenes. Here we have designed and characterized a library of synthetic promoters to facilitate multi-gene engineering efforts that enable the control of expression level and tissue-specificity in planta. By leveraging an orthogonal transcriptional system, we have developed a strategy that utilizes both synthetic activators and synthetic repressors to coordinate the simultaneous expression of several genes, introducing logic principles for genetic circuits and multiple layers of transcriptional control. Moreover, our characterization of the contributory genetic elements that dictate gene expression provides the foundation for future efforts in the rational design of more refined synthetic promoters. Our findings demonstrate how these promoters can enable the concerted expression of multiple genes simultaneously in a tissue-specific and environmental-responsive manner, providing the basis for more elegant and sophisticated plant engineering endeavors.

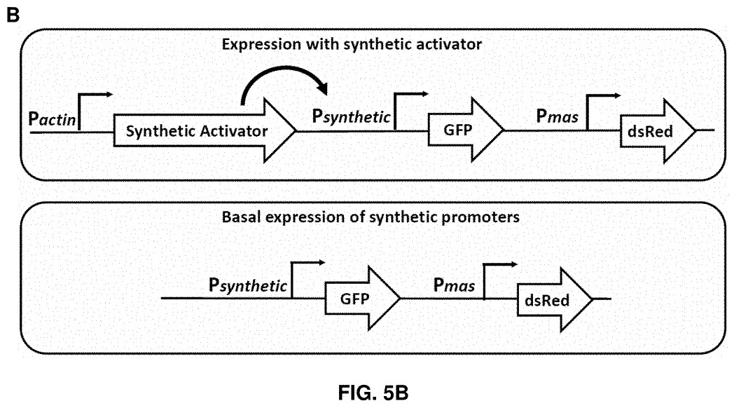

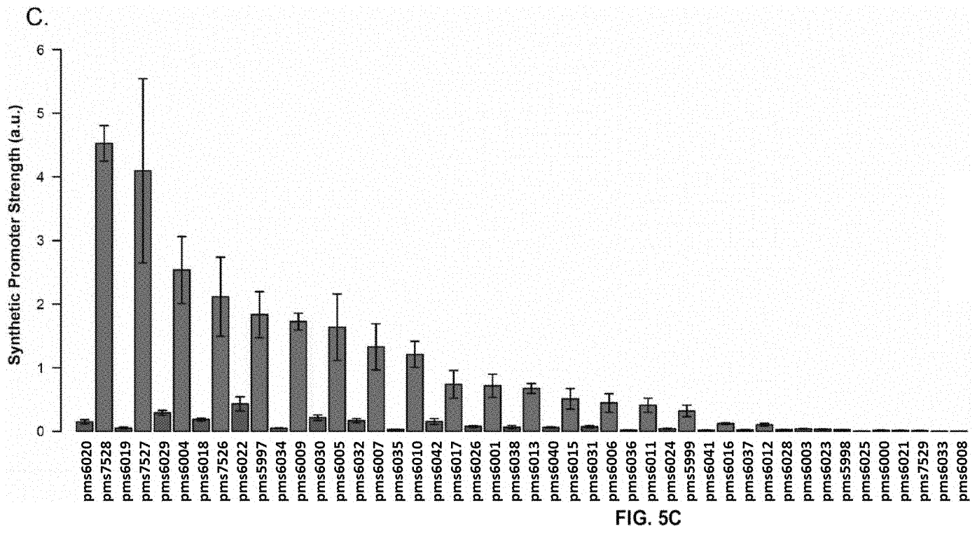

In order to develop a library of synthetic promoters that displays a diversity of varying expression strengths, we designed a system that would be turned on by an orthogonal transcription factor, hereon referred to as a synthetic activator. We utilized the well-characterized transcription factor Gal4, from Saccharomyces cerevisiae, fused to a VP16 activator domain as a synthetic activator¹⁰. The heterologous nature of Gal4, originating from a distantly related organism, provides a means to leverage a purely orthogonal system in plants, separating the transcriptional regulation of introduced transgenes from endogenous genes. A diversity of known Gal4 cis-elements—also known as upstream activating sequences (UAS)—were taken from endogenous promoters in the yeast Gal regulon, displaying distinct nucleotide sequences and assumed to exhibit a diversity of dissociation constants with Gal4. Each promoter was composed of five randomly chosen UASs upstream of a previously characterized plant minimal promoter^{11,12} (FIGS. 5A to 5C). Moreover, it has become increasingly recognized that the minimal promoter—i.e., the region in which RNA polymerase II is recruited via the TATA element to initiate transcription—also plays a key role in determining the expression strength of a given gene¹³.

By generating a random library fusing various constructed cis-element regions upstream of distinct minimal promoters, a diversity of expression strengths were generated, as measured via Green Fluorescent Protein (GFP) fluorescence and using DsRed driven by a constitutive promoter for normalization. In order to decrease variability from co-infiltrations of different agrobacterium strains, we stacked the genes into one binary vector so only one agrobacterium strain was infiltrated into tobacco leaves for characterization¹⁴. As expected, we observed a wide distribution of expression strengths, with the resulting pool of promoters spanning over a ten-fold range of protein levels (FIGS. 5A to 5C).

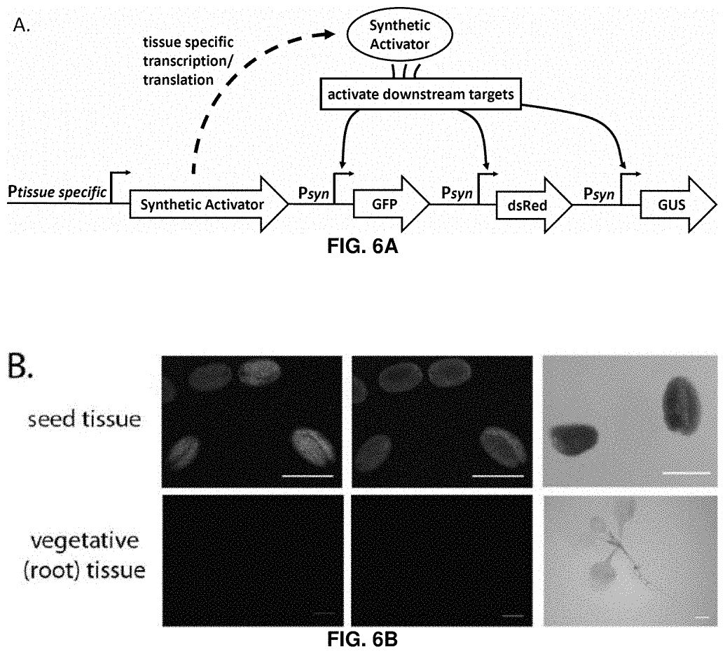

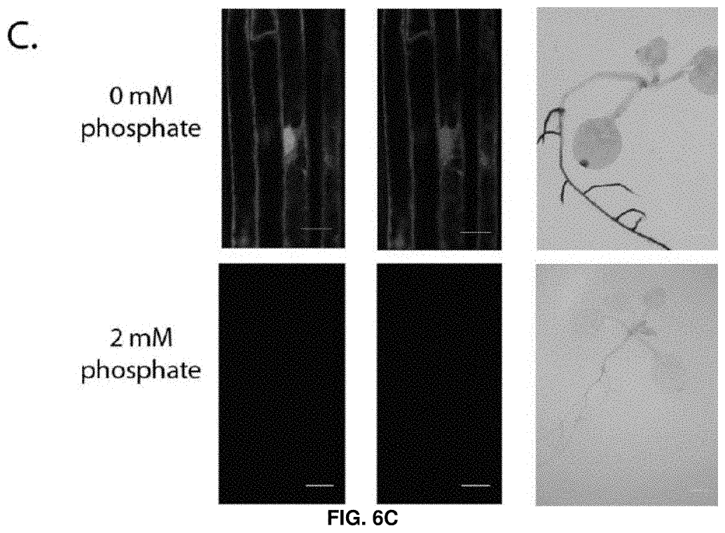

Inherent to our library design of synthetic promoters is the avoidance of identical sequences, as addressed by the randomization of varying cis-element combinations and minimal promoter sequences to avoid homologous sequences. This strategy permits engineering efforts to stack multiple genes together with the use of distinct promoters to avoid potential silencing issues that may be observed with the use of identical promoter sequences. In order to demonstrate that these genes can coordinate the expression of multiple genes in a tissue-specific manner, we expressed the synthetic activator under a seed-specific promoter, stacked with three reporter genes (GFP, DsRed, and beta-glucuronidase (GUS)) driven by three different synthetic promoters. As the reporters can only be turned on by the synthetic activator, we observed the expected expression of all three reporters specifically in seed tissue (FIGS. 6A to 6C). Our findings demonstrate the use of synthetic promoters in the spatial regulation of multiple stacked genes simultaneously.

In many cases, it is not necessary to express transgenes constantly, and thus temporal control over gene expression provides an additional dimension of control over engineering endeavors. For example, the constant overexpression of a given protein in an organism may act as a sink on cellular resources resulting in overall detrimental effects¹⁵. Similarly, various agricultural traits (e.g., disease resistance) can many times result in fitness costs, and thus targeted expression of these genes may curtail unintended consequences^{16,17} Thus, coordinating the expression of multiple genes in an inducible or environmentally-responsive manner may provide a solution to many plant engineering efforts. In order to highlight how our synthetic promoters may address some of these issues, we designed a synthetic activator driven by a phosphate responsive promoter, which is induced under low external phosphate concentrations, again stacked with three reporter genes driven by synthetic promoters. Stable Arabidopsis transformants display the expression of all reporter genes in response to the absence of phosphate in media (FIGS. 6A to 6C), and as expected, reporter genes were repressed in the presence of phosphate. These results display how our synthetic promoters may be leveraged to enable fine control over stacked genes in applications that require the complex engineering efforts that can respond to environmental cues (i.e., abiotic and biotic stresses).

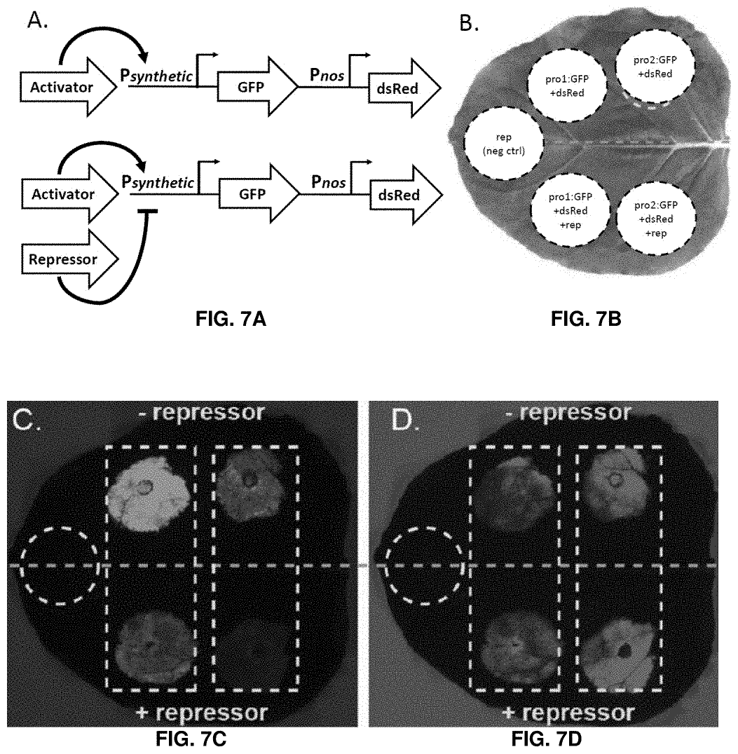

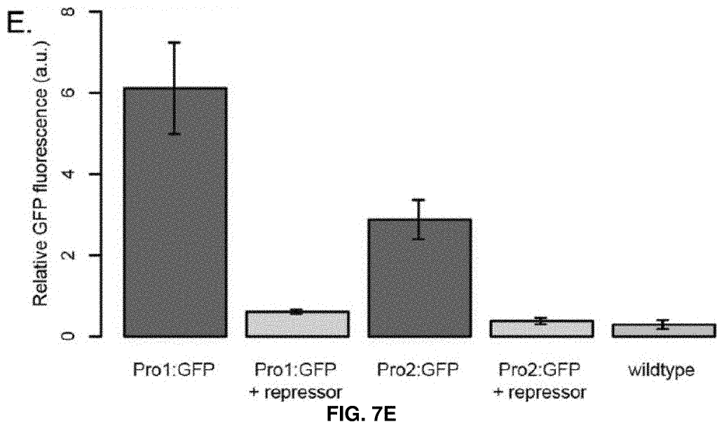

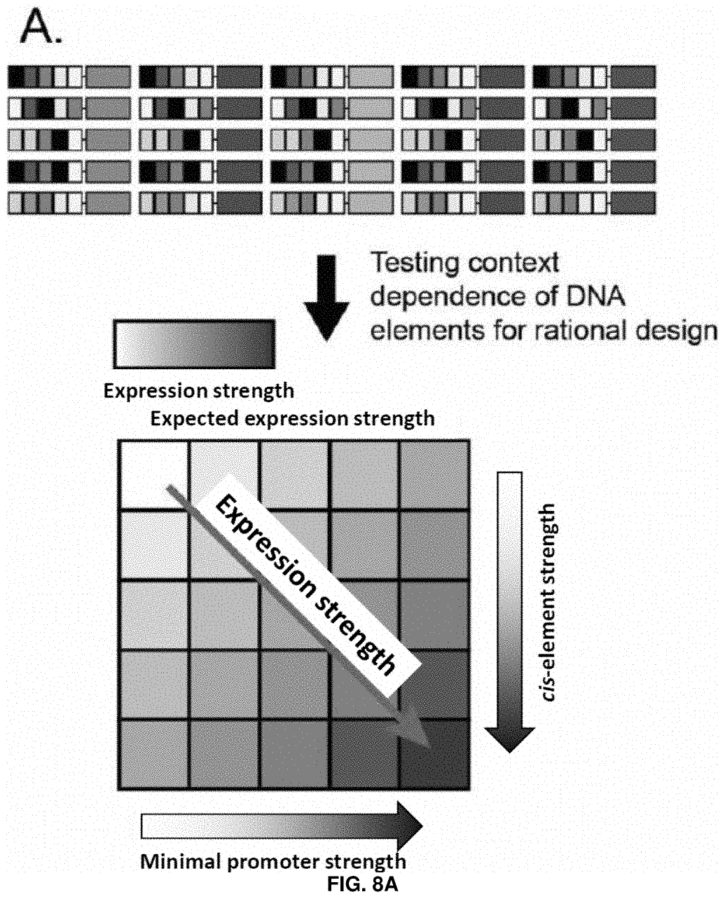

An additional level of control can be designed into our system with the introduction of other regulators, permitting logic to be built into genetic circuits. In order to explore this possibility, we designed synthetic repressors that bind to our synthetic promoters and repress transcription. As a proof of concept, three synthetic repressors were constructed by fusing a SRDX repressor domain to the Gal4 DNA-binding domain¹⁸. When synthetic repressors were co-infiltrated into tobacco leaves with synthetic promoters driving GFP expression, GFP fluorescence decreased, indicative of a repression in gene expression (FIGS. 7A to 7E). This proof-of-concept finding demonstrates another dimension of control over transgenes that is achieved through the addition of repressor logic into our system. For example, more complex engineering efforts may be pursued that could utilize repressor logic in a spatial manner, where various transgenes can be constitutively expressed, except in a specified tissue-type where the synthetic repressor is driven by a targeted promoter.

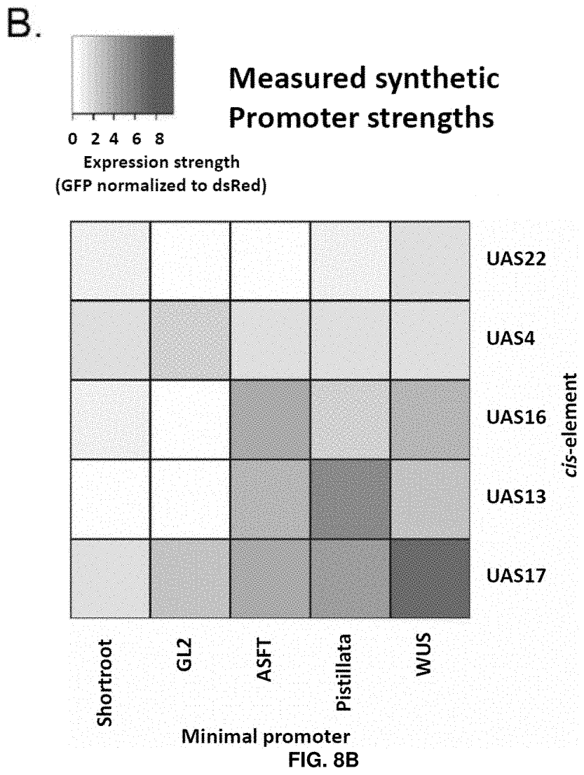

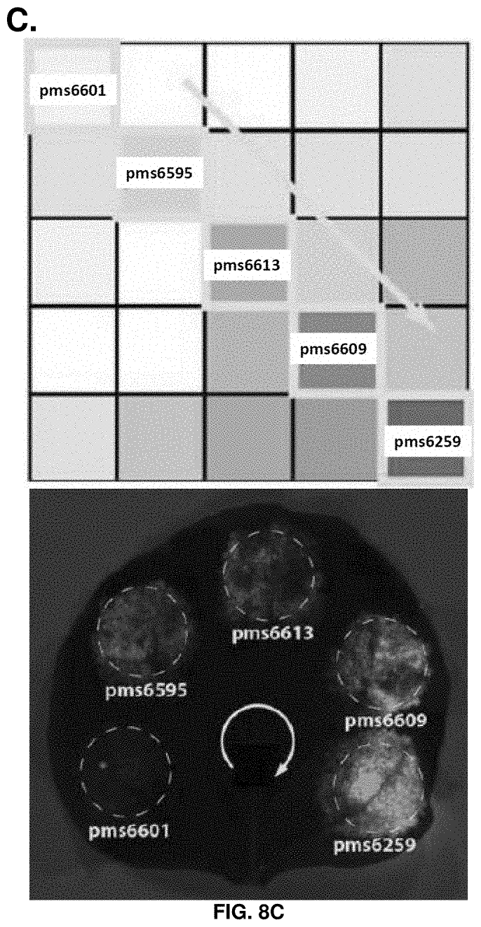

Our library of randomly designed synthetic promoters not only characterizes a distribution of expression strengths, but also provides the opportunity to begin empirically testing the genetic components that make up plant promoter architecture. The comparatively simple and better-characterized promoter structure of prokaryotes has enabled the design of various synthetic promoters^{3,19}; however, even in the most well studied eukaryotic systems (i.e., yeast), we are only beginning to elucidate the genetic components that dictate gene expression²⁰. In order to begin testing the contributions of various cis-elements and minimal promoter elements, we constructed a combinatorial library of five random cis-element sequences (each consisting of five concatenated UAS sequences) and five minimal promoters. All twenty-five possible combinations of cis-element and minimal promoter were generated, and the expression strength was measured via GFP fluorescence and normalized by to a constitutive promoter driving DsRed. The expression profile of all thirty-six promoters reveals an additive effect of promoter elements that contribute to overall expression strength. That is, cis-elements that have a higher affinity for the synthetic activator drive higher gene expression, and similarly, minimal promoters can be identified that increase expression strength as well (FIGS. 8A to 8C). Noise in this data is most likely due to genetic context. The empirical characterization of these synthetic promoters demonstrates the ability to rationally design and engineer synthetic promoters with predicted expression strengths.

It has been difficult to unravel the genetic determinants underpinning gene expression in eukaryotes, especially in plants. A core tenet of synthetic biology is the ability to understand the fundamental and reductionist rules that govern natural systems in order to reconstruct and engineer artificial molecular components of life. Our findings demonstrate the ability to tease apart the additive role of various cis-elements and minimal promoters that dictate gene expression, providing the foundation for future studies to rationally design promoters with expected expression strengths a priori. Although there are many nuisances to transcriptional regulation in plants that have not been elucidated in this study, our results take one step towards the coarse dissection of the contributory effects of specific genetic elements in controlling gene expression, and future studies may characterize and catalog cis-elements and minimal promoters that will permit the rational design of custom promoters. These findings may provide the foundation for the future identification of design principles that will enable the construction of more refined and targeted expression of transgenes in plants.

With increased global demand for food in the face of a growing population, there is pressing motivation and enthusiasm to engineer plants that tackle many impending societal challenges, such as abiotic stress, disease resistance, biofortification, and sustainability. In order to deliver on such agricultural biotechnology solutions, tools will need to be developed that address basic challenges in controlling transgene expression strength, enabling tissue-specific expression, and stacking multiple genes without risks of gene silencing. With these concerns in mind, we have designed a library of synthetic promoters with the intent of addressing a number of these issues and have demonstrated their utility in planta. Improving the capabilities of plant scientists to perform targeted and precise engineering will be indispensable for future complex multi-gene engineering efforts.

Methods:

Generation of constructs. In order to facilitate the large amount of DNA assembly needed for this study, all constructs described in this study were generated using the yeast assembly-based jStack method, previously described¹⁴. The use of yeast assembly enabled multiple the rapid assembly of various parts into pYB binary vectors, facilitating downstream experiments. Briefly, all the DNA parts were synthesized or cloned as individual DNA parts into starting plasmids (Level 0), with flanking BsaI cut sites. Cassettes composed of linkers, promoters, CDS, and terminator were assembled via Golden Gate cloning²¹, generating Level 1 constructs. Various Level 1 cassettes were linearized and transformed into yeast along with linearized pYB vector in order to facilitate the assembly of all cassettes into the binary vector via homologous stretches of DNA which overlap via Linker and Terminator sequences. All Level 2 constructs were assembled into the binary vector pYB2301¹⁴.

In total, over half a seven hundred thousand base pairs (701,847 bp) of DNA (including Level 0 and Level 1 intermediary shuttle vectors) were synthesized, cloned, and assembled in this study. In total, 461,006 bp of DNA were assembled into binary vectors in this study. All constructs are available through the JBEI ICE registry²².

Design of synthetic promoters. A library of UAS Gal4 binding cis-elements and minimal promoters were collected. In order to generate a library of promoters that displayed a broad distribution of expression strengths, five randomly chosen UAS sequences were concatenated together and fused to a random minimal promoter. UAS elements were taken from promoter regions of known genes in the Gal regulon and that have been previously identified based on the seventeen base pair binding motif 5′-CGG-NNNNNNNNNNN-CCG-3′, wherein N is any nucleotide (SEQ ID NO:1). Known minimal promoter from various plants were synthesized based on previous studies¹¹¹². DNA promoter parts were synthesized and cloned into the pUC57-Kan vector with flanking BsaI cut sites compatible with standardized Golden Gate²¹ and jStack DNA assembly methods¹⁴.

The synthetic activator was codon-optimized for Arabidopsis and synthesized where the DNA-binding domain of Gal4 was fused to a SV40 NLS and VP16 activator domain on the C-terminus. The synthetic activator was generated by swapping the VP16 activator domain for a SRDX repressor domain.

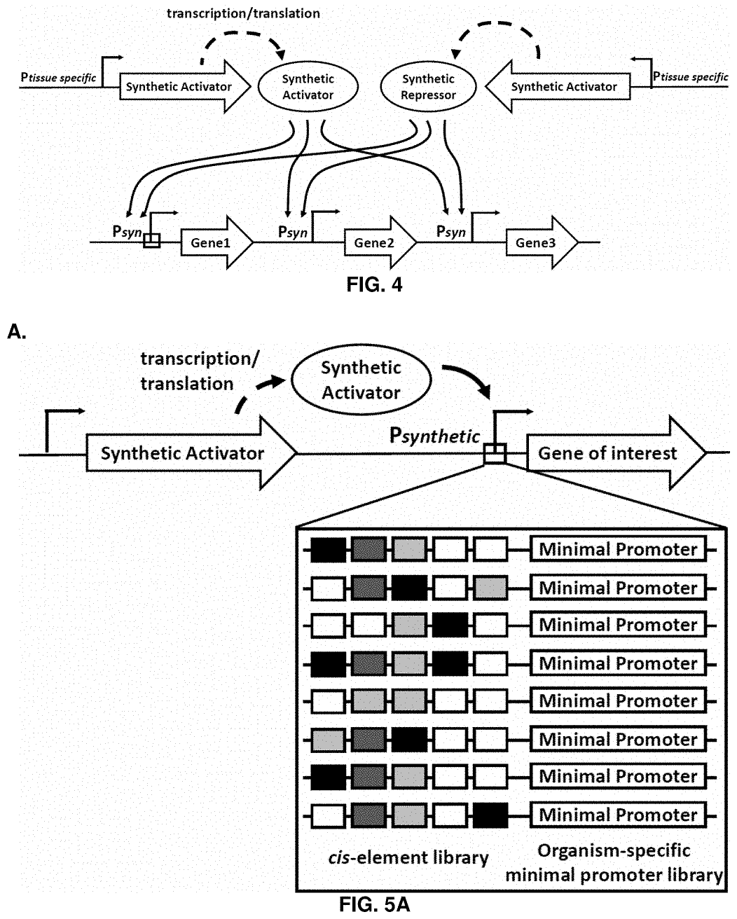

A second library of thirty-six synthetic promoters were designed and generated (FIG. 4) composed of combinations of various UAS and minimal promoter elements. Six concatenated UAS elements that were generated in the initial synthetic promoter library were chosen along with six minimal promoters. All thirty-six combinations of UAS element fused to a given minimal promoter were synthesized.

Characterization of synthetic promoters. All constructs stacked three basic cassettes: 1) a Gal4 synthetic activator constitutively driven by the Arabidopsis Actin2 promoter, 2) a synthetic promoter driving GFP, and 3) a DsRed constitutively driven by a MAS promoter. All plasmids were assembled into pYB binary vectors using the jStack yeast assembly method¹⁴. Constructs assembled into binary vectors were transformed into Agrobacterium tumefaciens strain GV3101. Transformed Agrobacterium strains were grown in liquid media with appropriate antibiotics and diluted to an OD600=1.0. Leaves of four week old Nicotiana benthamiana plants were infiltrated following the procedure described in Sparkes et al²³. Nicotiana benthamiana plants were grown and maintained in Percival-Scientific growth chambers at 25° C. in 16/8 hour light/dark cycles with 60% humidity. Leaves were collected four days after infiltration, and leaf disks were taken from leaves floated on 200 μL of water in 96 well microtitre plates, and GFP and DsRed fluorescence of each leaf disk was measured using a Synergy 4 microplate reader (Biotek). For each construct eight biological replicates (leaf disks) were taken. Samples were normalized by DsRed expression. Synthetic repressor experiments were measured just as described above, but Agrobacterium strains transformed with binary vectors containing synthetic repressors were co-infiltrated into Nicotiana benthamiana leaves.

Arabidopsis transformation. Promoters for the seed-specific (At2S3) and phosphate-inducible promoters (AtPht1.1) were previously cloned from Arabidopsis thaliana Col-0 genomic DNA¹⁴. Cassettes to drive the synthetic activator were built with either the At2S3 or AtPht1.1 promoter upstream of the activator. Reporter genes (GFP, DsRed, and GUS) were driven by synthetic promoters and all Level 1 cassettes were yeast assembled into the binary vector pYB2301 resulting in the final constructs pms5857 and pms6504 respectively.

The plasmids were each transformed into the Agrobacterium tumefaciens strain GV3101, which was subsequently used for transformation into Arabidopsis Col-0 background using the floral dip infiltration method²⁴. Transformed Arabidopsis plants were selected by plating the resulting T1 seeds onto agar plates containing ½ Murashige and Skoog (Phytotechlab, http://www.phytotechlab.com/), 1% (w/v) sucrose, and 50 μg mL-1 Kanamycin. After 2 weeks, resistant plants were moved to soil.

Plant growth conditions. Arabidopsis seeds were surface sterilized, rinsed with sterile water and stratified at 4° C. for 3 days. Seeds were germinated on agar plates with half-strength MS (½ MS) salts containing 1% sucrose with kanamycin resistance for 2 weeks. Seedlings were grown vertically on agar plates in growth chamber at 21-23° C. for 2 weeks with a light intensity of 100-130 μE m⁻² s⁻¹ under a short-day light cycle (10 h light/14 h dark).

Phosphate availability experiments.

For the phosphate experiments, seeds were grown under sterile conditions on vertical agar plates with one set containing half-strength MS (½ MS) salts lacking phosphate, iron and nitrogen (Phytotechlab, http://www.phytotechlab.com/) supplemented with nitrogen (5 mM ammonium nitrate) and iron (100 μM Fe-EDTA), to only lack phosphate content. Where seedlings were grown on no phosphate, the KH₂PO₄/K₂HPO₄ was replaced with KCl to maintain the potassium ion concentration in the medium. The other set were supplemented with all the above mentioned nutrients as well as 2 mM phosphate (KH₂PO₄) all containing 1% (w/v) sucrose with kanamycin resistance for 2 weeks.

For the phosphate experiments, the seeds were grown on ½ MS media agar plates (Phytotechlab, http://www.phytotechlab.com/) with (2 mM KH₂PO₄) and without phosphate with 1% (w/v) sucrose with kanamycin resistance for 2 weeks. Where seedlings were grown on no phosphate, the KH₂PO₄/K₂HPO₄ was replaced with KCl to maintain the potassium ion concentration in the medium.

Microscope analysis.

A laser scanning confocal microscope (LSM 710; Carl Zeiss Microscopy) was used for fluorescence analysis of Arabidopsis plants stably transformed with the reporter genes. Excitation of GFP and DsRed was performed using lasers at 488 with emission filter 510-530 nm and 558 nm with emission filter 583-592 nm, respectively 2 week old seedlings expressing GFP and DsRed were used for imaging.

Histochemical GUS staining.

To assay β-glucuronidase (GUS) reporter activity, whole DsRED- or GFP-positive seeds and seedlings were infiltrated with staining solution (1 mM EDTA, 0.2% Triton X-100, 0.2% Tween-20 in TBS, pH 7.3) containing 1 mM 5-bromo-4-chloro-3-indolyl-β-D-glucuronide (X-Gluc). Ferricyanide (0.25 mM) was added to prevent indigo precursor migration²⁵. The chelator EDTA was added to the staining solution to prevent any gene expression during the staining procedure. In seedlings, substrate penetration was assisted by two vacuum infiltrations at 0.1 atm for 15 min each on ice to improve infiltration. In seeds, substrate penetration was assisted by incubating around 20 seeds in round filter papers, moistened with water and placed in a plastic petri dish. After 3-day pre-chilling at 4° C. and 22 h incubation at 22° C., the small filter papers supporting the seeds were briefly blotted on dry filter papers to remove excessive water and subsequently GUS staining was carried out²⁶. The seedlings and seeds were incubated in staining solution at 37° C. until sufficient blue staining had been developed.

### REFERENCES CITED

1 Shih, P. M., Liang, Y. & Loqué, D. Biotechnology and synthetic biology approaches for metabolic engineering of bioenergy crops. Plant J 87, 103-117 (2016).
2 Salis, H. M., Mirsky, E. A. & Voigt, C. A. Automated design of synthetic ribosome binding sites to control protein expression. Nat Biotech 27, 946-950 (2009).
3 Mutalik, V. K. et al. Precise and reliable gene expression via standard transcription and translation initiation elements. Nat Meth 10, 354-360 (2013).
4 Hawkins, K. M. & Smolke, C. D. Production of benzylisoquinoline alkaloids in Saccharomyces cerevisiae. Nat Chem Biol 4, 564-573 (2008).
5 Potvin-Trottier, L., Lord, N. D., Vinnicombe, G. & Paulsson, J. Synchronous long-term oscillations in a synthetic gene circuit. Nature 538, 514-517 (2016).
6 Sunilkumar, G., Mohr, L., Lopata-Finch, E., Emani, C. & Rathore, K. S. Developmental and tissue-specific expression of CaMV 35S promoter in cotton as revealed by GFP. Plant Mol Biol 50, 463-479 (2002).
7 Liu, W. et al. Computational discovery of soybean promoter cis-regulatory elements for the construction of soybean cyst nematode-inducible synthetic promoters. Plant Biotechnology Journal 12, 1015-1026, doi:10.1111/pbi.12206 (2014).
8 Fagard, M. & Vaucheret, H. (Trans)Gene silencing in plants: How Many Mechanisms? Annu Rev Plant Physiol Plant Mol Biol 51, 167-194 (2000).
9 Matzke, M. A., Primig, M., Trnovsky, J. & Matzke, A. J. M. Reversible methylation and inactivation of marker genes in sequentially transformed tobacco plants. EMBO J 8, 643-649 (1989).
10 Sadowski, I., Ma, J., Triezenberg, S. & Ptashne, M. GAL4-VP16 is an unusually potent transcriptional activator. Nature 335, 563-564 (1988).
11 Kiran, K. et al. The TATA-Box Sequence in the Basal Promoter Contributes to Determining Light-Dependent Gene Expression in Plants. Plant Physiol 142, 364-376 (2006).
12 Joshi, C. P. An inspection of the domain between putative TATA box and translation start site in 79 plant genes. Nucleic Acids Research 15, 6643-6653 (1987).
13 Lubliner, S. et al. Core promoter sequence in yeast is a major determinant of expression level. Genome Res 25, 1008-1017 (2015).
14 Shih, P. M. et al. A robust gene-stacking method utilizing yeast assembly for plant synthetic biology. Nature Comm 7, 13215 (2016).
15 Harcum, S. W. & Bentley, W. E. Heat-shock and stringent responses have overlapping protease activity in Escherichia coli. Implications for heterologous protein yield. Appl Biochem Biotechnol 80, 23-37 (1999).
16 Tian, D., Traw, M. B., Chen, J. Q., Kreitman, M. & Bergelson, J. Fitness costs of R-gene-mediated resistance in Arabidopsis thaliana. Nature 423, 74-77 (2003).
17 Denancé, N., Sánchez-Vallet, A., Goffner, D. & Molina, A. Disease resistance or growth: the role of plant hormones in balancing immune responses and fitness costs. Front Plant Sci 4, 155 (2013).
18 Hiratsu, K., Matsui, K., Koyama, T. & Ohme-Takagi, M. Dominant repression of target genes by chimeric repressors that include the EAR motif, a repression domain, in Arabidopsis. Plant J 34, 733-739 (2003).
19 Jensen, P. R. & Hammer, K. The Sequence of Spacers between the Consensus Sequences Modulates the Strength of Prokaryotic Promoters. Appl Environ Microbiol 64, 82-87 (1998).
20 Levo, M. & Segal, E. In pursuit of design principles of regulatory sequences. Nat Rev Genet 15, 453-468 (2014).
21 Patron, N. J. et al. Standards for plant synthetic biology: a common syntax for exchange of DNA parts. New Phytol 208, 13-19, doi:10.1111/nph.13532 (2015).
22 Ham, T. S. et al. Design, implementation and practice of JBEI-ICE: an open source biological part registry platform and tools. Nucleic Acids Res 40, e141-e141 (2012).
23 Sparkes, I. A., Runions, J., Kearns, A. & Hawes, C. Rapid, transient expression of fluorescent fusion proteins in tobacco plants and generation of stably transformed plants. Nat Protocols 1, 2019-2025 (2006).
24 Clough, S. J. & Bent, A. F. Floral dip: a simplified method for Agrobacterium-mediated transformation of Arabidopsis thaliana. Plant J 16, 735-743 (1998).
25 Jefferson, R. A. Assaying chimeric genes in plants: The GUS gene fusion system. Plant Mol Biol Reporter 5, 387-405 (1997).
26 Liu, P.-P., Koizuka, N., Martin, R. C. & Nonogaki, H. The BME3 (Blue Micropylar End 3) GATA zinc finger transcription factor is a positive regulator of Arabidopsis seed germination. Plant J 44, 960-971 (2005).

All of the references cited herein are hereby incorporated by reference.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A genetically modified plant cell or plant, comprising: (a) (i) one or more nucleic acids each encoding one or more transcription factors (or transcription activators) operatively linked to a first tissue-specific or inducible promoter, (ii) one or more nucleic acids each encoding one or more transcription repressors each operatively linked to a second tissue-specific or inducible promoter, or (iii) combinations thereof; and (b) one or more nucleic acids each encoding one or more independent genes of interest (GOI) each operatively linked to a promoter that is activated by the one or more transcription factors (or transcription activators), repressed by the one or more transcription repressors, or a combination of both.

2. The genetically modified plant cell or plant of claim 1, wherein the transcription factor (or transcription activator) is eukaryotic or prokaryotic.

3. The genetically modified plant cell or plant of claim 1, wherein the transcription factor (or transcription activator) is synthetic.

4. The genetically modified plant cell or plant of claim 1, wherein the transcription repressor is synthetic.

5. The genetically modified plant cell or plant of claim 1, wherein the transcription factor (or transcription activator) and the transcription repressor are synthetic.

6. The genetically modified plant cell or plant of claim 1, wherein any one of the transcription factor (or transcription activator), plant cell or plant, one or more of the GOI, any other transcription factor (or transcription activator), transcription repressor, and/or any of the promoters is heterologous to any other member of the list herein.

7. The genetically modified plant cell or plant of claim 6, wherein the transcription factor (or transcription activator) is heterologous to the plant cell or plant, one or more of the GOI, any other transcription factor (or transcription activator), transcription repressor, and/or any of the promoters.

8. The genetically modified plant cell or plant of claim 6, wherein the transcription repressor is heterologous to the plant cell or plant, one or more of the GOI, any other transcription factor (or transcription activator), and/or any of the promoters.

9. The genetically modified plant cell or plant of claim 1, wherein the genetically modified plant cell or plant comprises: (a) a first nucleic acid encoding a transcription factor (or transcription activator) operatively linked to a first tissue-specific or inducible promoter, (b) optionally a second nucleic acid encoding a transcription repressor operatively linked to a second tissue-specific or inducible promoter; and (c) one or more nucleic acids each encoding one or more independent genes of interest (GOI) each operatively linked to a promoter that is activated by the transcription factor (or transcription activators), repressed by the transcription repressors, or a combination of both.

10. The genetically modified plant cell or plant of claim 1, wherein the genetically modified plant cell or plant comprises: (a) optionally a first nucleic acid encoding a transcription factor (or transcription activator) operatively linked to a first tissue-specific or inducible promoter, (b) a second nucleic acid encoding a transcription repressor operatively linked to a second tissue-specific or inducible promoter; and (c) one or more nucleic acids each encoding one or more independent genes of interest (GOI) each operatively linked to a promoter that is activated by the transcription factor (or transcription activators), repressed by the transcription repressors, or a combination of both.

11. The genetically modified plant cell or plant of claim 1, wherein each GOI is operatively linked to a promoter that is activated by the transcription factor (or transcription activator), repressed by the transcription repressors, or a combination of both.

12. The genetically modified plant cell or plant of claim 11, wherein the promoter comprises one or more DNA-binding sites specific for the transcription factor (or transcription activator), one or more DNA-binding sites specific for the transcription repressor, or a combination of both.

13. The genetically modified plant cell or plant of claim 1, wherein the promoter comprises 1 to 10 DNA-binding sites specific for the transcription factor (or transcription activator), 1 to 10 DNA-binding sites specific for the transcription repressor, or a combination of both.

14. A library of unique promoters, wherein the promoter strengths of every unique promoter is identified relative to every other unique promoter.

15. The library of claim 14, wherein the library comprises at least 8 unique promoters.

16. The library of claim 15, wherein the library comprises at least 10 unique promoters.

17. The library of claim 16, wherein the library comprises at least 20 unique promoters.

18. The library of claim 17, wherein the library comprises at least 50 unique promoters.

19. The library of claim 18, wherein the library comprises at least 100 unique promoters.

20. The library of claim 14, wherein each unique promoters has had the promoter strength of each promoter tested and compared to every other unique promoter, such that the promoter strengths of every unique promoter is identified relative to every other unique promoter, and the unique promoters can be ordered according to descending or ascending promoter strength.
